(19) United States
(12) Patent Application Publication
(10) Pub. No.: US 2017/0247696 A1
(43) Pub. Date: Aug. 31, 2017

(54) MICRORNA WITH PLACENTA-PERMEABILITY AND USES THEREOF

(71) Applicant: JIANGSU MICROMEDMARK BIOTECH CO., LTD., Jiangsu (CN)

(72) Inventors: Chenyu Zhang, Jiangsu (CN); Jing Li, Jiangsu (CN); Yujing Zhang, Jiangsu (CN); Ke Zeng, Jiangsu (CN)

(21) Appl. No.: 15/523,323

(22) PCT Filed: Oct. 27, 2015
(86) PCT No.: PCT/CN2015/092974

§ 371 Date: Apr. 28, 2017

(30) Foreign Application Priority Data
Oct. 28, 2014 (CN) 201410591247.5

(51) Int. Cl. C12N 15/113

(52) U.S. Cl. CPC C12N 15/113; C12N 2310/141; C12N 2310/14

(57) ABSTRACT

The present invention discloses the use of placenta permeable small RNAs, particularly the use of small ribonucleic acids (small RNAs) in the preparation of a medicament comprising the small RNAs as active substances, wherein the medicament is administered to a mother's body directly and the small RNAs enter the fetus through the placenta to play their role in the fetus. Trials have shown that the small RNAs when administered directly to the mother's body in different ways such as oral and intragastric administration can enter the fetus through the placenta to play their role directly in the fetus. The invention provides a novel administration method that can minimize the damage to a fetus.

### TECHNICAL FIELD

The present invention pertains to the use of placenta permeable small RNAs, and in particular, to the small RNAs or a substance containing the small RNAs being directly administered to a mother's body in different ways to enter the fetus through the placenta to play its desired effect.

### BACKGROUND ART

The placenta is an important organ formed during pregnancy, which is an interface between the maternal and fetal circulatory systems, through which the fetus can obtain nutrients and oxygen from the mother's body and discharge his/her metabolic wastes; besides, the placenta plays a role of barrier for selective exchange of materials. Specifically, the barrier separating the maternal and fetal circulatory systems consists of four layers: the fetal vascular endothelial cells, trophoblast, cytotrophoblast, and syncytiotrophoblast respectively. Generally it is believed that a substance from a mother's body must pass through the barrier composed of the four layers of cells before entering the fetal circulatory system. Studies have shown that, during a material transport process, oxygen, carbon dioxide and many small molecules are transported through diffusion and osmosis; large molecules such as proteins, antibodies, hormones, etc. are transported through active transport and pinocytosis; glucose, the primary energy source for fetus, passes through the placenta through facilitated diffusion; the fetal blood amino acids with a concentration higher than that in maternal blood come out of the placenta through active transport; the maternal free fatty acids pass through the placenta through free diffusion; some metal ions such as sodium and potassium (Na+, K+), vitamins and metabolic inhibitors can pass through the placenta through active transport; and some larger molecules (such as Rh positive antigen, etc.) are generally unable to be transported, but may be cross-flowed and mixed under the circumstances of oxygen deficit, trauma and special delivery.

Studies have shown that maternal nutrients can enter the fetus through the placenta, and some harmful substances ingested by the mother may also influence the growth and development of the fetus through the placenta. Occurrence of many fetal congenital diseases is closely associated with the external environmental factors on the mother. Intrauterine infection, i.e. maternal pathogens entering a fetus through placental tissues, is an important factor that causes fetal malformations, abortion or stillbirth. During pregnancy, especially in the first three months, mothers undergo a series of physiological changes, one of which is immunosuppression, turning down the resistance of mothers against a variety of microorganisms, causing increased susceptibility to pathogen infections and reactivation of latent viruses in the body. During this kind of infection, pregnant women themselves sense no subjective symptoms and only show viremia. However, such intrauterine infection caused by various viruses and pathogens may cause permanent injury to a fetus, including embryonic death, abortion, stillbirth, various congenital malformations, etc. Pathogenic microorganisms that may lead to fetal intrauterine infection during pregnancy include Toxoplasma gondii, herpes simplex virus, cytomegalovirus, rubella virus, etc.

The fetus's immune response does not expresses itself until about 23 weeks of pregnancy, therefore if a mother is infected with teratogenic pathogens, should the fetus's infections be identified until and after the 23rd week of pregnancy by drawing cord blood to detect the related virus antibody or DNA. At this time, it is likely necessary to perform labor induction if infection is determined, which will cause great physical and psychological harm to the mother.

If labor induction is not performed, the existing treatment methods for intrauterine infections are as follows: the acyclovir, ganciclovir or adenosine arabinoside therapies for herpes simplex virus infection, but with certain toxic and side effects; the penicillin therapy for congenital syphilis, wherein cephalosporin can be used if there is allergy to penicillin, but lack of specificity; the sulfadiazine, pyrimethamine or spiramycin therapies for toxoplasmosis, but having some teratogenic effects; and intramuscular injection of human polyclonal immunoglobulin for rubella virus, but its efficacy remains to be confirmed. In addition, medications during pregnancy have inevitable risks of teratogenesis.

Therefore, it is urgent to have an effective and safe method for treatment of intrauterine infection and developmental defects caused thereby.

Contents of the Invention

One object of the present invention is to provide the use of small ribonucleic acids (small RNAs, referred to as small RNAs hereinafter) for preparation of a medicament.

Another object of the present invention is to provide a method for administration of the small RNAs on a fetus.

Still another object of the present invention is to provide a kit for detection of small RNAs.

One aspect of the present invention provides the use of small RNAs including micro-ribonucleic acids (miRNAs), small interfering ribonucleic acids (siRNAs) or a combination thereof for preparation of a medicament, wherein the medicament comprises the small RNAs as active substances, the medicament is administered to a mother's body directly and the small RNAs enter the fetus through the placenta to play their role in the fetus.

In another preferred embodiment, the administration includes oral, intravenous and subcutaneous injection.

In another preferred embodiment, the small RNAs include miRNAs, siRNAs, or a plasmid carrying miRNAs/siRNAs.

In another preferred embodiment, the small RNAs comprise of a single stranded nucleic acid sequence or its complementary sequence.

In another preferred embodiment, the micro-ribonucleic acids (mircoRNAs, referred to as miRNAs hereinafter) are miRNAs of a plant, an animal and a microorganism, or artificially synthetic miRNAs, or a combination thereof.

In another preferred embodiment, the miRNAs are miRNAs derived from a plant.

In another preferred embodiment, the miRNAs include miRNAs that can inhibit viral infection or replication.

In another preferred embodiment, the miRNAs include miRNA2911 from honeysuckle.

In another preferred embodiment, the small interference ribonucleic acids (small interference RNAs, referred to as siRNAs hereinafter) is derived from siRNAs of a plant, an animal and a microorganism, or artificially synthetic siRNAs, or a combination thereof.

In another preferred embodiment, the medicament is used for preventing and treating a fetal disease, or regulating the normal growth and development of a fetus.

In another preferred embodiment, the medicament comprises 0.1-99.99 wt % of a pharmaceutically acceptable carrier and 0.01-99.9 wt % of the small RNAs.

In another preferred embodiment, the small RNAs include modified or unmodified miRNAs, modified or unmodified siRNAs or a combination thereof.

In another preferred embodiment, the fetal disease includes congenital diseases (such as heart, liver, metabolic and other diseases), intrauterine infectious diseases during pregnancy (such as diseases caused by rubella virus, herpes virus or other microbial infections) and fetal distress caused by intrauterine distress.

In another preferred embodiment, the infectious diseases include infections caused by the following pathogens: viruses, bacteria, mycoplasma and chlamydia.

In another preferred embodiment, the viruses include rubella virus and herpes virus.

A second aspect of the present invention provides a method of administering small RNAs to a fetus, comprising the following steps:

(a) administering small ribonucleic acids (small RNAs) to a mother's body so that the small RNAs can enter the fetus from the mother's body through the placenta to play their role in the fetus, wherein the small RNAs include micro-ribonucleic acids (miRNAs), small interference ribonucleic acids (siRNAs) or a combination thereof.

In another preferred embodiment, the administering as described in step (a) includes oral administration, inhalation, intragastric administration, blood injection, intramuscular injection, or a combination thereof.

In another preferred embodiment, the term “enter the fetus” as described in step (a) means that the small RNAs enter a fetus' blood, plasma, serum, body fluids, cells, tissues, organs, or combinations thereof.

In another preferred embodiment, the tissues include fetal liver tissues.

In another preferred embodiment, the method further comprises a step of:

(b) detecting the presence or absence of the small RNAs from the maternal and/or fetal samples and the content thereof, wherein the samples include blood, plasma, serum, body fluids, cells, tissues, organs, or combinations thereof.

In another preferred embodiment, the tissues include fetal liver tissues.

A third aspect of the present invention provides a kit used for the administration of small RNAs, comprising:

(a) a container and a medicament in the container, wherein the medicament comprises the small RNAs as active substances, and when the medicament is administered to a mother's body directly, the small RNAs enter the fetus through the placenta to play their role in the fetus; and

(b) an instruction for use.

In another preferred embodiment, the instruction describes the following administration method: the medicament is administered to a mother's body and the small RNAs enter the fetus through the placenta to play their role in the fetus.

In another preferred embodiment, the instruction further describes that the medicament is used for preventing and treating a fetal disease, or regulating the normal growth and development of a fetus.

It should be understood that all of the various technical features described above and specifically described hereinafter (such as embodiments) can be combined with one another within the scope of the present invention, so as to form new or preferred technical solutions. Due to space limitations, these are no longer tired out one by one.

### DESCRIPTION OF DRAWINGS

The following drawings are intended to illustrate specific embodiments of the invention rather than limit the scope of the present invention as defined by the claims.

FIG. 1 shows the results of deep sequencing of plant-derived micro-ribonucleic acids in human umbilical cord blood and amniotic fluid.

FIG. 2 shows the miR2911 level of a honeysuckle extract in the sera of pregnant mice (A: fold change; B: concentration). FIG. 2A shows the fold change of miR2911 of a honeysuckle extract in the sera of pregnant mice; and FIG. 2B shows the concentration of miR2911 of a honeysuckle extract in the sera of pregnant mice.

FIG. 3 shows the miR2911 level of a honeysuckle extract in the livers of fetal mice.

FIG. 4 shows the level of synthetic siRNAs in the sera of pregnant mice (A: fold change; B: concentration). FIG. 4A shows the fold change of the synthetic siRNAs in the sera of pregnant mice; and FIG. 4B shows the concentration of the synthetic siRNAs in the sera of pregnant mice.

FIG. 5 shows the level of synthetic siRNAs in the livers of fetal mice.

FIG. 6 shows the mRNA level of α-fetoprotein in the livers of fetal mice.

FIG. 7 shows an inhibitory effect of the siRNAs on HCMV virus in vitro.

FIG. 8 shows that the siRNAs enter the fetal mouse through a maternal mouse to produce an inhibitory effect on the rubella virus in vivo.

### PARTICULAR EMBODIMENTS

The inventor(s), after extensive and deep studies, by administering different sources of exogenous small RNAs to mother's bodies, firstly found that small RNAs can enter the fetus through the placenta to regulate the gene expression in the fetus, influencing the growth and development and disease states of the fetus. On this basis, the present invention is completed.

Small Ribonucleic Acids (Small RNAs)

In the present invention, the term “small ribonucleic acids (small RNAs)” refers to small RNA fragments with a length of more than twenty nucleotides; according to the widely accepted classification method proposed by Steven Buckingham in May 2003, small RNAs (small RNAs) refer to the part of non-coding RNAs other than transcriptional RNAs (including ribosomal RNAs and transfer RNAs), and includes microRNAs, short interference RNAs (siRNAs), small nucleolar RNAs (snoRNAs), small nuclear RNAs (snRNAs), etc.

Micro-ribonucleic acids (microRNAs, miRNAs) are a type of single-stranded small ribonucleic acid molecules with a length of about 19-23 nucleotides; they are located at the non-coding region of a genome and evolutionarily highly conserved, and may regulate the gene expression by inhibiting the translation process of target genes and are closely related with many normal physiological activities of animals, such as the individual development, tissue differentiation, cell apoptosis, and energy metabolism of organisms, and meanwhile, they are closely associated with the occurrence and development of many diseases. The existing studies have further confirmed that plant-derived miRNAs can also be ingested into animals to involve in regulatory activities.

Small interfering ribonucleic acids (small interfering RNAs, siRNAs) are a type of double-stranded RNA molecules composed of more than 20 nucleotides, which can exert the effect of silencing gene expression by specifically degrading the messenger ribonucleic acids (messenger RNAs, mRNAs) of target genes. This process is called RNA interference, RNAi. RNA interference is a mode of post-transcriptional regulation of genes. siRNAs can specifically recognize target genes thereof and can recruit a protein complex which is called silencing complex (RNA induced silencing complex, RISC). RISC comprises ribonuclease and the like, which can specifically and efficiently inhibit the expression of genes through targeted cleavage of homologous mRNAs. Since the use of RNA interference technology can specifically knock out or shut down the expression of particular genes, this technology has been widely used in the fields of biomedical experimental researches and therapies of various diseases.

Kit

The present invention further provides a kit used for the administration of small RNAs, comprising:

(a) a container and a medicament in the container, wherein the medicament comprises the small RNAs as active substances, and when the medicament is administered to a mother's body directly, the small RNAs enter the fetus through the placenta to play their role in the fetus; and

(b) an instruction for use.

In another preferred embodiment, the instruction describes the following administration method: the medicament is administered to a mother's body and the small RNAs enter the fetus through the placenta to play their role in the fetus.

In another preferred embodiment, the instruction further describes that the medicament is used for preventing and treating a fetal disease, or regulating the normal growth and development of a fetus.

The Main Advantages of the Present Invention Lie in that:

(1) the fact that small RNAs can enter the fetus through the placenta for the first time is revealed;

(2) a method to regulate gene expression in the fetus and influence the fetal growth and development by administering small RNAs to a mother's body to enter the fetus is provided;

(3) by administering small RNAs to a mother's body, such as injecting a small

RNA-loaded plasmid and other manners, the small RNAs enter the fetus through the placenta to play their role directly, and the damage to the fetus is minimized; and

(4) a method for researching and developing new drugs for treating intrauterine fetal diseases is provided.

The present invention is further illustrated in connection with particular embodiments as follows. It should be understood that these embodiments are merely illustrative of the invention and are not intended to limit the scope of the present invention. In the case of specific conditions for an experimental method being not specified in the following examples, generally conventional conditions are followed, such as the conditions described in Sambrook et al., Molecular Cloning: A Laboratory Manual (New York: Cold Spring Harbor Laboratory Press, 1989), or in the Plant Molecular Biology-A Laboratory Mannual, edited by Melody S. Clark, Springer-verlag Berlin Heidelberg, 1997), or the conditions recommended by the manufacturer are followed. All percentages and parts are by weight unless otherwise indicated.

### EXAMPLE 1

### The Plant-Derived Micro-Ribonucleic Acids Pass Through Maternal Placenta

1.1 Collection and Detection of Umbilical Cord Blood and Amniotic Fluid.

Reagents: Trizol was purchased from Invitrogen Corporation; reverse transcriptase and Taq enzyme were purchased from Dalian Baocheng Bioengineering Co., Ltd.; micro-ribonucleic acid probes were purchased from Invitrogen Corporation.

Amniotic fluid was collected and every 10 samples were mixed into a group, and extracted for RNAs for deep sequencing. Umbilical cord blood was collected and centrifuged at 3000 rpm for 15 minutes, and the serum supernatant was collected. Every 10 samples were mixed into a group, and extracted for RNAs for deep sequencing.

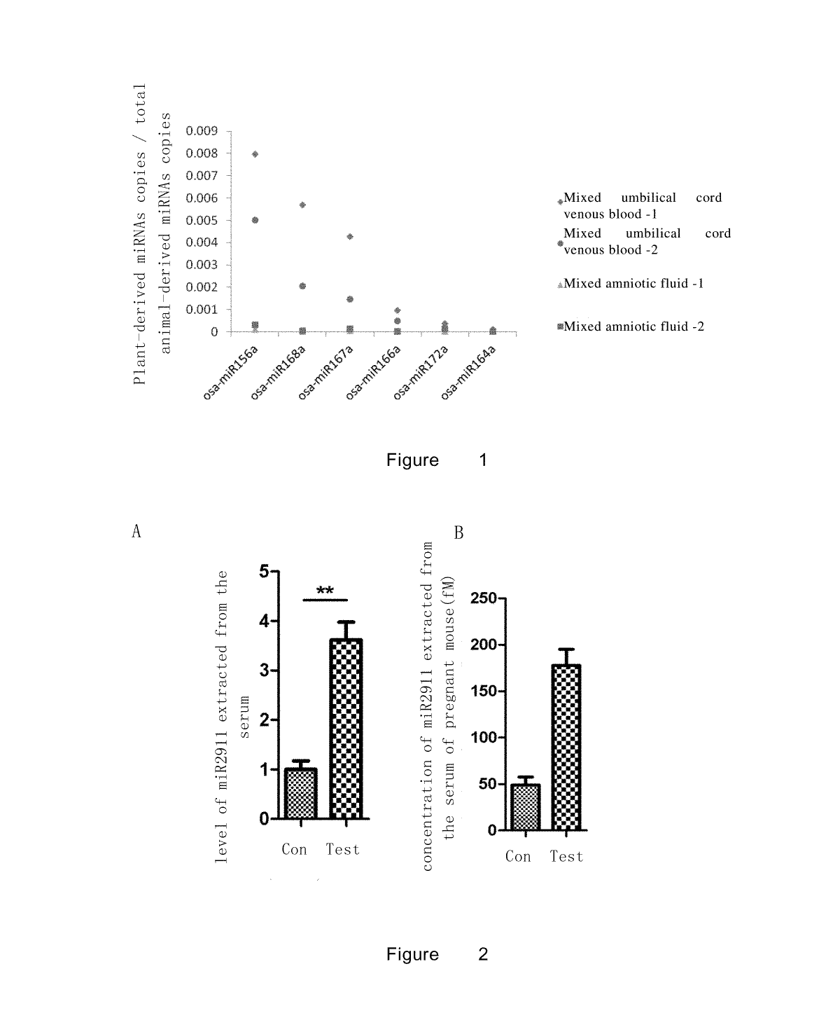

The particular result is shown as in FIG. 1. FIG. 1 shows the results of deep sequencing of plant-derived micro-ribonucleic acids in human umbilical cord blood and amniotic fluid. The total copy number of the animal micro-ribonucleic acids was taken as a correction, and the plant-derived micro-ribonucleic acids including osa-miR156a, osa-miR168a, osa-miR167a, osa-miR166a, osa-miR172a, and osa-miR164a from rice were detected. It can be seen from FIG. 1 that the presence of plant-derived micro-ribonucleic acids osa-miR156a, osa-miR168a, osa-miR167 a, osa-miR166a, osa-miR172a, and osa-miR164 a can be detected in human umbilical cord blood and amniotic fluid, suggesting that the plant-derived micro-ribonucleic acids can pass through the maternal placenta. The sequences of the above-mentioned miRNAs can be obtained by searching from the public database miRB ase.

| | SEQ | |
|---|---|---|
| | ID | |
| miRNA name | NO.: | sequence (5′-3′) |
| osa-MIR156a | 1 | UGACAGAAGAGAGUGAGCAC |
| osa-MIR168a | 2 | UCGCUUGGUGCAGAUCGGGAC |
| osa-MIR167a | 3 | UGAAGCUGCCAGCAUGAUCUA |
| osa-MIR166a | 4 | GGAAUGUUGUCUGGUUCAAGG |
| osa-MIR172a | 5 | AGAAUCUUGAUGAUGCUGCAU |
| osa-MIR164a | 6 | UGGAGAAGCAGGGCACGUGCA |

### EXAMPLE 2

### Plant-Derived Micro-Ribonucleic Acids Enter the Fetus Through the Maternal Placenta

Honeysuckle was purchased from Simcere Pharmaceutical;Trizol was purchased from Invitrogen Corporation; reverse transcriptase and Taq enzyme were purchased from Dalian Baocheng Bioengineering Co., Ltd.; and micro-ribonucleic acid probes were purchased from Invitrogen Corporation.

2.1 Preparation of an aqueous extract of honeysuckle: 25 ml of water per gram of honeysuckle was added, and simmered for 30 min, then the soup was collected and concentrated to 1 g honeysuckle extract/ml.

2.2 Animal Experiments

C57/BL6J mice, SPF grade, were purchased from Model Animal Center of Nanjing University, and the animals were fed according to the standard. One male mouse and two female mice were mated in one cage, on the next morning, the vaginal plugs of the female mice were checked and if the plug was seen, mating was successful; and in 14 days after pregnancy, experiments were carried out when the placenta was mature. The Honeysuckle extract was administered intragastrically at a dose of 1 ml of honeysuckle extract per pregnant mouse, and 3 hours later, pregnant mice were treated. Blood was taken from the eyeballs, centrifuged at 3000 rpm to collect serum; the fetal mice were separated, and after carefully washing, fetal liver tissues were separated and preserved in liquid nitrogen. For the control group, an equal volume of normal saline was administered intragastrically. 3 hours later, pregnant mice were treated. Blood was taken from the eyeballs, centrifuged at 3000 rpm to collect serum; the fetal mice were separated, and after carefully washing, fetal liver tissues were separated and preserved in liquid nitrogen.

2.3 Extraction of Serum RNAs and Real-Time PCR Detection

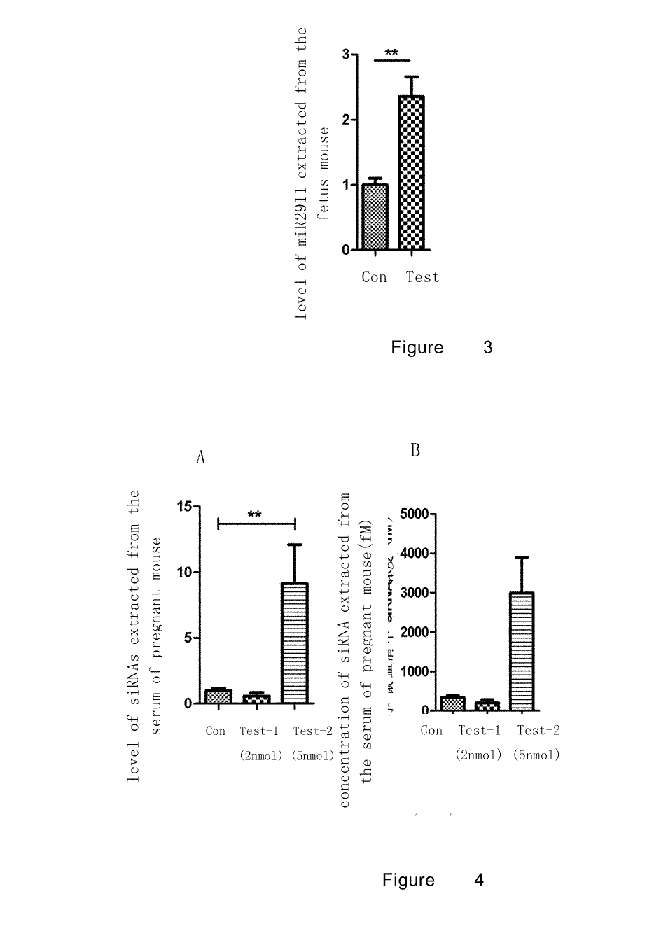

100 μL of serum was taken, to which 300 μL of DEPC water was added, mixed uniformly, then 200 μL of water saturated phenol was added, and after vigorous shaking, 200 μL of chloroform was added and shaken well, centrifuged at 16000 g at room temperature for 15 min; after centrifugation was finished, the supernatant (about 400 μL) was carefully drawn and 2-fold volume of isopropyl alcohol was added, stood at −20° C. for 60 min, then centrifuged at 16000 g at 4° C. for 20 min; after centrifugation was finished, the supernatant was discarded and 75% ethanol was added, mixed by inverting gently, centrifuged at 16000 g at 4° C. for 20 min; after centrifugation was finished, the supernatant was discarded and 20 μL of DEPC water was added to dissolve. 2 μl of RNAs was taken for reverse transcription, followed by taking 1 μl of cDNA for real-time PCR detection of the expression of target small nucleic acids. The particular result is shown as in FIG. 2-FIG. 3. FIG. 2 shows the miR2911 level of a honeysuckle extract in the sera of pregnant mice. FIG. 2A shows the fold change of miR2911 of a honeysuckle extract in the sera of pregnant mice; and FIG. 2B shows the concentration of miR2911 of a honeysuckle extract in the sera of pregnant mice. Control group: intragastric administration of normal saline with an equal volume as in the experiment group (n=f8); experiment group: intragastric administration of a honeysuckle extract, with a dose of 1 g of honeysuckle extract per mouse (n=8). It can be seen from FIG. 2 that the miR2911 expression level in the sera of pregnant mice was increased significantly in the experiment group as compared with the control group, suggesting that miR2911 in the honeysuckle extract entered the circulatory system of the pregnant mice through the intestinal tract of the pregnant mice. FIG. 3 shows the miR2911 level of a honeysuckle extract in the livers of fetal mice. Control group: intragastric administration of normal saline with an equal volume as in the experiment group (n=16); experiment group: intragastric administration of a honeysuckle extract, with a dose of 1 g of honeysuckle extract per mouse (n=16). It can be seen from FIG. 3 that the miR2911 expression level in the livers of fetal mice was increased significantly in the experiment group as compared with the control group, suggesting that miR2911 in the sera of pregnant mice entered the fetal liver tissues through the placenta.

### EXAMPLE 3

### Artificially Synthetic Small Interfering Nucleic Acids (siRNAs) of α-Fetoprotein Highly Expressed in the Fetus Period can Pass Through the Placenta and Regulate the Expression of α-Fetoprotein mRNAs

siRNAs were purchased from Invitrogen Corporation; α-fetoprotein mRNAs were purchased from Invitrogen Corporation, and micro-ribonucleic acid probes were purchased from Invitrogen Corporation; Trizol was purchased from Invitrogen Corporation; reverse transcriptases and Taq enzymes were purchased from Dalian Baocheng Biological Engineering Co., Ltd.

In this example, the artificially synthetic small interfering nucleic acids (siRNAs) of a-fetoprotein (AFP) highly expressed in the fetus period was used for tracing, demonstrating that the small interfering nucleic acid could pass through the placenta and regulate the expression of a-fetoprotein mRNAs.

The experimental pre-treatment of the experimental mice was as described in Example 2. Experiment was started when the placenta became mature after 14 days of pregnancy. In the experiment group, mice were intragastrically administered at a dose of 2 nmol/mouse and 5 nmol/mouse; at the same time, mice in the control group were intragastrically administered with an equal volume of normal saline. 3 hours later, mice were treated, blood was taken from eyeballs and collected, and fetal mice were taken for separation of liver tissues.

3.1 Extraction of Tissue RNAs and Real-Time PCR Detection

An appropriate amount of tissues were taken, to which 1 ml of Trizol was added, ground to dispersion of tissues with a grinding rod, then 200 μL of chloroform was added, shaken acutely, centrifuged at 12000 g at 4° C. for 15 min; after centrifugation was finished, the supernatant was carefully drawn, an equal volume of isopropyl alcohol was added, stood at room temperature for 10 min, then centrifuged at 12000 g at 4° C. for 10 min; after centrifugation was finished, the supernatant was discarded and 1 mL of 75% ethanol was added, then centrifuged at 12000 g at 4° C. for 5 min; after centrifugation was finished, the supernatant was discarded and an appropriate amount of DEPC water was added to dissolve. 1 μg of RNAs were taken for reverse transcription, followed by taking 1 μl of cDNAs for real-time PCR detection of the expressions of target small nucleic acids and mRNAs, and specific results were shown as in FIG. 4-FIG. 6.

FIG. 4 shows the level of the synthetic siRNAs in the sera of pregnant mice. FIG. 4A shows the fold change of the synthetic siRNAs in the sera of pregnant mice; and FIG. 4B shows the concentration of the synthetic siRNAs in the sera of pregnant mice. Control group: intragastric administration of notmal saline with an equal volume as in the experiment group (n=4); experiment group 1: intragastric administration of a siRNA solution at a dose of 2 nmol (n=4); experiment group 2: intragastric administration of an siRNA solution at a dose of 5 nmol (n=4).

It can be seen from FIG. 4 that the expression level of the synthetic siRNAs in the sera of pregnant mice was significantly increased at an intragastric dose of 5 nmol/mouse as compared with the control group. However, when the intragastric dose was 2 nmol/mouse, the expression level of the synthetic siRNAs in the sera of pregnant mice was not changed significantly, suggesting that the exogenous siRNAs can be absorbed by the intestinal tract to enter circulatory systems of the pregnant mice.

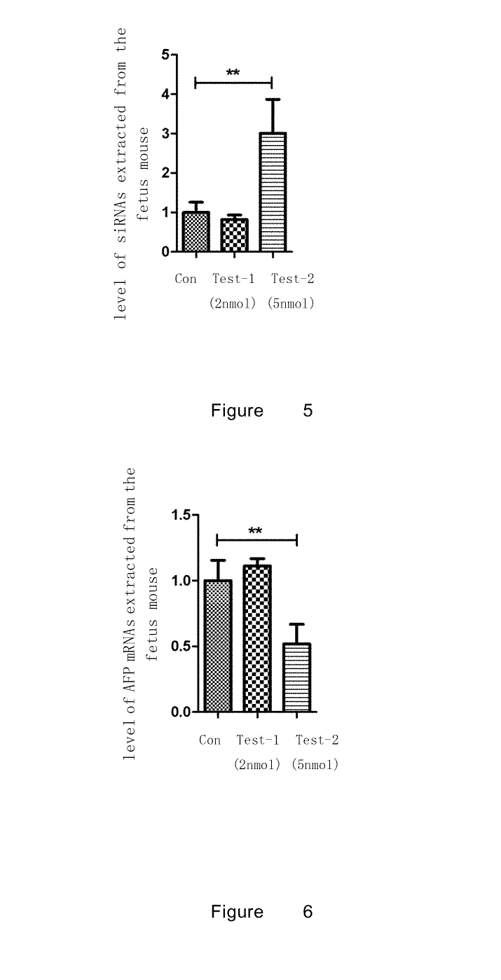

FIG. 5 shows the level of synthetic siRNAs in the livers of fetal mice. Control group: intragastric administration of notmal saline with an equal volume as in the experiment group (n=8); experiment group 1: intragastric administration of an siRNA solution at a dose of 2 nmol (n=8); experiment group 2: intragastric administration of an siRNA solution at a dose of 5 nmol (n=8).

It can be seen from FIG. 5 that when the intragastric dose was 5 nmol/mouse, the level of the synthetic siRNAs was significantly increased in the liver tissues of fetal mice as compared with the control group; however, when the intragastric dose was 2 nmol/mouse, the expression level of the synthetic siRNAs was not changed significantly in the liver tissues of fetal mice. It was suggested that the siRNAs in pregnant mice can pass through the placenta to enter the liver tissues of fetal mice; however, the siRNAs were insufficient to be detected in fetal mice after intragastric administration at a low dose, and only when the intragastric dose was increased, can siRNAs that entered the liver tissues of fetal mice be adequate to be detected.

FIG. 6 shows the mRNA level of α-fetoprotein in the livers of fetal mice. Control group: intragastric administration of normal saline with an equal volume as in the experiment group (n=8); experiment group 1: intragastric administration of an siRNA solution at a dose of 2 nmol (n=8); experiment group 2: intragastric administration of an siRNA solution at a dose of 5 nmol (n=8).

It can be seen from FIG. 6 that the level of a-fetoprotein mRNAs was significantly decreased at an intragastric dose of 5 nmol/mouse as compared with the control group. However, when the intragastric dose was 2 nmol/mouse, the level of a-fetoprotein mRNAs was not changed significantly, suggesting that siRNAs entered the liver tissues of mice to exert its biological functions, reduced the level of a-fetoprotein mRNAs in the livers of fetal mice and influenced the gene expression of the fetus, thereby further affecting the biological processes such as liver development.

### EXAMPLE 4

### Inhibition of Human Cytomegalovirus (HCMV) using SiRNAs

The main content of this example was an in vitro trial, to validate the effective inhibition of human cytomegalovirus using siRNAs.

The specific experiment steps were as follows:

1) Human embryonic lung fibroblasts (imported IMR-90, purchased from Nanjing KeyGEN BioTECH Co., Ltd.) were cultured in DMEM media at a constant temperature of 37° C., and mixed with 10% FBS and 1% penicillin uniformly.

2) HCMV AD169 virus stain was purchased from CGMCC. Human embryonic lung fibroblasts were infected with HCMV AD169 virus at an effective infection amount for use in subsequent experiments.

3) Design of siRNAs

For the cDNA sequence of the main protein gene UL123 of HCMV (Gene ID: 3077513 retrieved by the GENEBANK database of the National Library of the United States), the sequence at 122 bp at the downstream of the initiation codon (SEQ ID NO.7: GGCTTGAGGGAAGGCACATAACT) was selected as the target sequence.

Target sequence of siRNAs:

| | |
|---|---|
| GGCTTGAGGGAAGGCACATAACT | (SEQ ID NO. 7) |

Designed siRNA sequence:

| | |
|---|---|
| Sense strand: | |
| 5′-CUUGAGGGAAGGCACAUAAUU-3′ | (SEQ ID NO. 8) |
| Antisense strand: | |
| 5′UUAUGUGCCUUCCCUCAAGUU-3′ | (SEQ ID NO. 9) |

4) The infected cells were collected 48 hours after virus infection, and then treated with the siRNAs. The control group was established. Samples were taken at a fixed time interval to detect the virus titer.

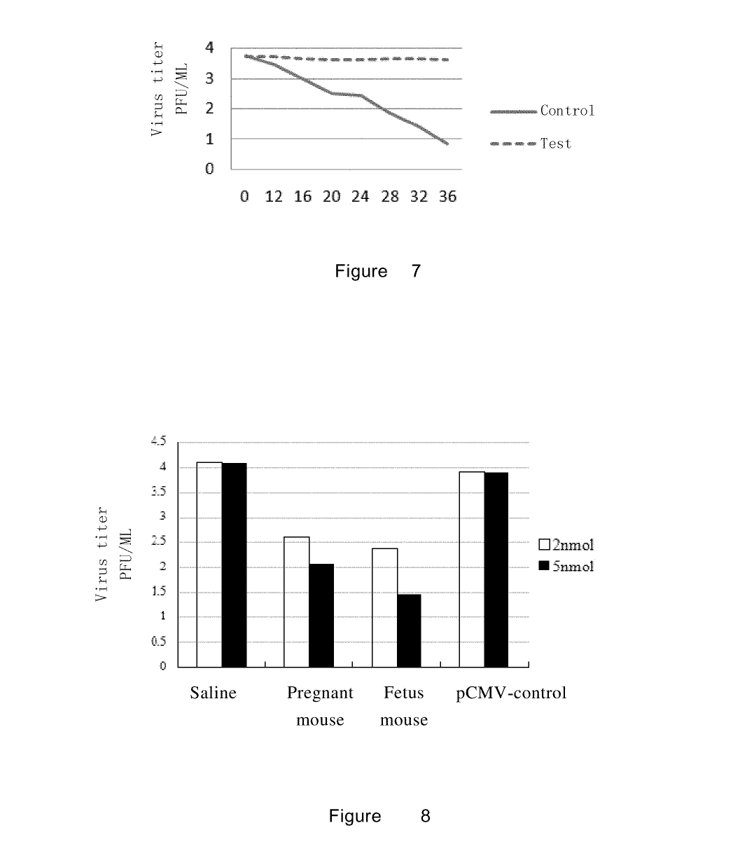

Experimental results were shown as in FIG. 7. It can be seen from FIG. 7 that the virus titer in the infected cells treated by the specific siRNAs were gradually decreased over the treatment time, and it can be seen therefrom that the specific siRNAs can effectively reduce the infection of HCMV virus. This example demonstrated that the specific siRNAs provided in the invention can function specifically for human cytomegalovirus, and play an antiviral role in human cell lines.

### EXAMPLE 5

### Inhibition of Rubella Virus using SiRNAs

In this example, small interfering RNAs were designed for the key gene E1 antigen gene fragment of rubella virus, and mice were administered by injecting a siRNA-loaded plasmid. Finally, it was demonstrated that the siRNAs can pass through the placenta to reach the fetal mice and play an anti-virus effect.

5.1 Design of Rubella Virus Specific SiRNAs

Small interfering RNAs were designed for the noncoding region of the key gene SL4 of rubella virus. Studies have shown that the inhibition of 3′ terminal noncoding region of SL4 of rubella virus can effectively inhibit the virus activity. The cDNA sequence of the small interfering RNAs was designed for the noncoding region of SL4 (Gene ID: M74327.1 retrieved in the GENEBANK database of the National Library of the United States), and the sequence at 482 bp at the downstream of the initiation codon

(SEQ ID NO.10: GACGACCATTATCGTTCAGATAA) was selected as the target sequence.

Target sequence of siRNAs:

| | |
|---|---|
| GACGACCATTATCGTTCAGATAA | (SEQ ID NO. 10) |

The designed siRNA sequence was named siRNA-RV herein:

| | |
|---|---|
| Sense strand: | |
| 5′-CGACCAUUAUCGUUCAGAUUU-3′ | (SEQ ID NO. 11) |
| Anti-sense strand: | |
| 5′-AUCUGAACGAUAAUGGUCGUU-3′ | (SEQ ID NO. 12) |

5.2 Inoculation with Rubella Virus in Pregnant Mice

Reagents and laboratory animals: Trizol was purchased from Invitrogen Corporation; reverse transcriptase and Taq enzyme were purchased from Dalian Baocheng Bioengineering Co., Ltd.; siRNAs were purchased from Invitrogen Corporation; micro-ribonucleic acid probes were purchased from Invitrogen Corporation; the experimental animal C57/BL6J mice were purchased from Model Animal Center of Nanjing University; rubella virus strain M15240 was purchased from CGMCC.

C57/BL6J mice, SPF grade, were purchased from Model Animal Center of

Nanjing University, and the animals were fed according to the standard. One male mouse and two female mice were mated in one cage, on the next morning, the vaginal plugs of the female mice were checked and if the plug was seen, mating was successful; and in 14 days after pregnancy, experiments were carried out when the placenta was mature. 40 pregnant mice were divided into four groups, 10 animals for each group, and all mice in the four groups were injected with rubella virus intravenously at 14 days of pregnancy, then the subsequent experiment was started 48 hours after the course of disease.

5.3 SiRNA Treatment for Pregnant Mice

The main experimental materials:

Pentobarbital sodium, syringes, artificially constructed plasmids over-expressing the siRNAs as described in part 5.1 (pCMV-siRNA-RV) (provided by School of Life Sciences, Nanjing University), lymphocyte separating solution (Tianjin Hao Yang Biological Manufacture Co., Ltd.).

Experimental method and steps:

5.3.1 Construction of a Plasmid Over-Expressing SiRNA-RV (pCMV-siRNA-RV)

The siRNA-RV fragment was ligated to the PacI-digested pAdTrack-CMV vector by restriction enzyme digestion and ligation, and after identification by sequencing, the correct clone was named pCMV-siRNA-RV. At the same time, an empty vector plasmid pCMV-control (control plasmid) was prepared as a negative control.

5.3.2 Animal Experiments:

In Group 1, each mouse was injected with 100 μL of pCMV-siRNA-RV at a concentration of 20 nmol/mL by tail vein injection till the final concentration of 2 nmol/mouse; in Group 2, each mouse was injected with 100 μL of pCMV-siRNA-RV at a concentration of 50 nmol/mL by tail vein injection till the final concentration of 5 nmol/mouse; in Group 3, each mouse was injected with 100 μL of pCMV-control at a concentration of 20 nmol/mL by tail vein injection till the final concentration of 2 nmol/mouse; and in Group 4, each mouse was injected with normal saline by tail vein injection, and 24 h later, pregnant mice were scarified. Blood was taken from the pregnant mice, and fetal mice were separated to take blood after carefully washing, and then preserved in liquid nitrogen.

5.4 Detection of Virus Titers of Pregnant Mice and Fetal Mice

The virus titer mean in the sera- of pregnant mice and fetal mice obtained in step 5.3 was detected, and results were shown in FIG. 8. It can be seen from FIG. 8 that specific siRNAs can effectively reduce the in vivo virus titer in pregnant mice, and pass through the placenta barrier to fetal mice. In addition, the concentration gradient experimental results showed that, the anti-viral effect of the siRNAs was concentration-dependent. It can be seen therefrom that the present invention provides an effective method against rubella virus during pregnancy.

Examples 4 and 5 further demonstrate that micro-ribonucleic acids can enter the fetus through the placenta. During pregnancy, exogenous micro-ribonucleic acids in the maternal diet can enter the fetus and regulate the gene expression in the fetus, thereby affecting the growth and development of the fetus, which suggests that the maternal diets play an important role in regulating epigenetics of the fetus. The present invention provides a new standard for the evaluation of dietary quality of pregnant women. The invention provides the use of endogenous small RNAs of pregnant mothers and small RNAs from food and microorganisms, which can pass through the placenta, as nutritional supplements for pregnant women. The maternal small RNAs during pregnancy can pass through the placenta, which will not only affect the intrauterine growth and development and possible diseases of the fetus, but may also affect the postnatal development and diseases. The invention provides a new potential method for the treatment of intrauterine fetal diseases.

Discussion

Micro-ribonucleic acids (microRNAs, miRNAs) are a class of small molecule nucleic acids attracting extensive attention in recent ten years. Studies have shown that micro-ribonucleic acid molecules can be stably present in the serum, with the species universality. Previous studies by the present inventor(s) have confirmed that humans and animals can ingest micro-ribonucleic acids from plants through diets. Exogenous plant micro-ribonucleic acids are absorbed in the intestinal tract after entering the intestinal tract, and wrapped in microvesicles in intestinal epithelial cells. These microvesicles carrying exogenous plant micro-ribonucleic acids are secreted into the circulatory system, so that the exogenous micro-ribonucleic acids are carried into tissues where the exogenous micro-ribonucleic acids regulate the expression of target genes and regulate biological functions of human and animals. On this basis, the concept that miRNAs are a new kind of nutrients is proposed.

In view of that micro-ribonucleic acids can be present in different types of organisms and have significant biological functions, it is known by detection through biological means in the present invention that the endogenous small RNAs of pregnant mothers and exogenous small RNAs from food and microorganisms can enter the fetus through the placenta, and affect the growth and development of the fetus by regulating the fetal genes. These small RNAs can not only affect the intrauterine growth and development and possible diseases of the fetus, but also may affect postnatal development and diseases.

The present invention provides a novel method for the treatment of fetal diseases caused by intrauterine infection by providing medicaments and methods for specifically targeting and regulating these small RNAs.

In addition, the present invention further provides a new standard for the evaluation of dietary quality of pregnant women.

The diets of pregnant women are also important for fetal growth and development. At present, the dietary balance index (DBI) evaluation system is used to measure if the dietary intake of pregnant women is reasonable in china. If the dietary intake cannot maintain the balance of nutrient intake, nutritional supplements can be used for regulation. However, this evaluation system is defective because miRNAs are not taken into account. For example, some plant foods are rich in miRNAs that are unfavorable to fetal growth and development; however, in this system, these plants are only sources of vitamins and cellulose, and thus not recommended to avoid intake for pregnant women.

The present invention further provides the use of small RNAs that can pass through the placenta as a nutritional supplement for pregnant women.

Through biological means, the present invention studies that endogenous small RNAs of the pregnant mothers and small RNAs from foods and microorganisms can enter the fetus through the placenta and have biological functions. On this basis, the present invention solves the following technical problems:

1. defect for the treatment of intrauterine infectious diseases of fetuses; and

2. lack of a new standard for the evaluation of dietary quality of pregnant women.

All the documents mentioned in the present invention are incorporatedly referred to, as well as each alone. In addition, it should be understood that after reading the teachings of the present invention described above, a skilled person in the art can make various changes or modifications of the invention, and these equivalent forms shall also fall into the scope of the present application as defined by the appended claims.

1.-6. (canceled)

7. A method for administering small RNAs to a fetus, characterized in that the method comprises the following step:
(a) administering small ribonucleic acids (small RNAs) to a mother's body so that the small RNAs can enter the fetus from the mother's body through placenta to play their role in the fetus, wherein the small RNAs include micro-ribonucleic acids (miRNAs), small interference ribonucleic acids (siRNAs) or a combination thereof.

8. The method of claim 7, characterized in that the term “enter the fetus” as described in step (a) means that the small RNAs enter a fetus' blood, plasma, serum, body fluids, cells, tissues, organs, or combinations thereof.

9. The method of claim 7, characterized in that the method further comprises the following step:
(b) detecting the presence or absence of the small RNAs from maternal and/or fetal samples and content thereof, wherein the samples include blood, plasma, serum, body fluids, cells, tissues, organs, or combinations thereof.

10. A kit used for the administration of small RNAs, characterized in that the kit comprises:
(a) a container and a medicament in the container, wherein the medicament comprises the small RNAs as active substances, and when the medicament is administered to a mother's body directly, the small RNAs enter fetus through placenta to play their role in the fetus; and
(b) an instruction for use.

11. The method of claim 7, characterized in that the small RNAs is used for preventing and treating a fetal disease, or regulating normal growth and development of a fetus.

12. The method of claim 11, characterized in that the fetal disease includes congenital diseases, intrauterine infectious diseases during pregnancy or fetal distress caused by intrauterine distress.

13. The method of claim 12, characterized in that said congenital diseases comprise heart, liver, or metabolic diseases.

14. The method of claim 12, characterized in that intrauterine infectious diseases during pregnancy comprise diseases caused by rubella virus, herpes virus.

15. A method for preventing and treating a fetal disease, or regulating normal growth and development of a fetus comprising steps of :
administering effective amounts of medicament comprising small RNAs as active substances to a mother's body so that the small RNAs can enter the fetus from the mother's body through placenta to play their role in the fetus, wherein the small RNAs include miRNAs, siRNAs or a combination thereof.

16. The method of claim 15, characterized in that the fetal disease includes congenital diseases, intrauterine infectious diseases during pregnancy or fetal distress caused by intrauterine distress.

17. The method of claim 15, characterized in that said congenital diseases comprise heart, liver, or metabolic diseases.

18. The method of claim 15, characterized in that intrauterine infectious diseases during pregnancy comprise diseases caused by rubella virus, herpes virus.


SEQUENCE LISTING (12 sequences)

SEQ ID NO: 1 - RNA, 20 nt, Oryza sativa
ugacagaagagagugagcac

SEQ ID NO: 2 - RNA, 21 nt, Oryza sativa
ucgcuuggugcagaucgggac

SEQ ID NO: 3 - RNA, 21 nt, Oryza sativa
ugaagcugccagcaugaucua

SEQ ID NO: 4 - RNA, 21 nt, Oryza sativa
ggaauguugucugguucaagg

SEQ ID NO: 5 - RNA, 21 nt, Oryza sativa
agaaucuugaugaugcugcau

SEQ ID NO: 6 - RNA, 21 nt, Oryza sativa
uggagaagcagggcacgugca

SEQ ID NO: 7 - DNA, 23 bp, artificial sequence
  oligonucleotides
ggcttgagggaaggcacataact

SEQ ID NO: 8 - RNA, 21 nt, artificial sequence
  oligonucleotides
cuugagggaaggcacauaauu

SEQ ID NO: 9 - RNA, 21 nt, artificial sequence
  oligonucleotides
uuaugugccuucccucaaguu

SEQ ID NO: 10 - DNA, 23 bp, artificial sequence
  oligonucleotides
gacgaccattatcgttcagataa

SEQ ID NO: 11 - RNA, 21 nt, artificial sequence
  oligonucleotides
cgaccauuaucguucagauuu

SEQ ID NO: 12 - RNA, 21 nt, artificial sequence
  oligonucleotides
aucugaacgauaauggucguu